(19) United States
(12) Patent Application Publication
(10) Pub. No.: US 2023/0190973 A1
(43) Pub. Date: Jun. 22, 2023

(54) METHODS AND APPPRATUS FOR DISINFECTION OF SURFACES

(71) Applicant: Purdue Research Foundation, West Lafayette, IN (US)

(72) Inventors: Luciano Castillo, Carmel, IN (US); Tanya Purwar, West Lafayette, IN (US); Victor Castano, Queretaro (MX); Ali Doosttalab, Cohoes, NY (US)

(21) Appl. No.: 18/066,661

(22) Filed: Dec. 15, 2022

Related U.S. Application Data
(60) Provisional application No. 63/290,474, filed on Dec. 16, 2021.

(51) Int. Cl. A61L 2/18; A61L 2/26; A61L 2/14

(52) U.S. Cl. CPC A61L 2/18; A61L 2/26; A61L 2/14; A61L 2101/30

(57) ABSTRACT

A disinfectant sprayer assembly, including a tank defining a volume, a disinfectant suspension contained within the volume, a nozzle having a size variable spray opening, a pump operationally connected to the volume and to the nozzle for pumping disinfectant from the volume through the spray opening to apply disinfecting to a predetermine surface having a predetermined set of physical characteristics for a period of time, an ion generator operationally connected to the volume for ionizing the disinfectant suspension, and a microprocessor operationally connected to the size variable spray opening. The ion generator voltage, spray opening size, and distance between the predetermined surface and the spray opening are predetermined based on the predetermined set of physical characteristics of the surface.

### CROSS-REFERENCE TO RELATED APPLICATIONS

This patent application claims priority to co-pending U.S. Provisional Patent App. Ser. No. 63/290,474, filed on 16 Dec. 2021, which is incorporated herein by reference.

### TECHNICAL FIELD

The present disclosure relates generally to electrochemistry, and, more particularly, to methods and apparati for disinfecting surfaces, especially frequently touched indoor and outdoor surfaces. Further, the disclosure relates to disinfecting surfaces utilizing electrostatic spray optimized based on surface characteristics.

### BACKGROUND

This section introduces aspects that may help facilitate a better understanding of the disclosure. Accordingly, these statements are to be read in this light and are not to be understood as admissions about what is or is not prior art.

The United States Centers for Disease Control and Prevention (US-CDC), published that up to 650,000 deaths, annually, are associated with respiratory diseases. Respiratory viruses, novel or mutated, have been a cause of medical conundrum for centuries now. In recent decades, the world has seen epidemics and pandemics caused by respiratory infections like Severe Acute Respiratory Syndrome coronavirus (SARS-CoV-1), Middle East Respiratory Syndrome (MERS) coronavirus, influenza virus and so on. In December 2019, the first case of novel Severe Acute Respiratory Syndrome coronavirus 2 (SARS-CoV-2) disease was reported. Commonly addressed as COVID-19 infection, COVID-19 was soon declared a pandemic. Since then, many researchers have attempted to quantify the pathways that lead to the transmission of this deadly virus so as to curb its rapid spread. There are different routes of inter-human virus transmission. Such infectious agents can be spread through direct or indirect contact, droplets, aerosol transmission, and/or the like. Direct human to human contact, or via intermediate fomites, aerosols or droplets generated during coughing, sneezing, breathing, or talking, that might remain suspended in air or settle on environmental surfaces, are the multiple means of this vector spread. With the current global pandemic, we have seen rising awareness about social distancing, wearing masks, washing hands and disinfecting our surroundings. However, direct contact is the predominant route of virus transmission, and it is difficult to quantify indirect routes of spread through fomites. This leads to difficulty in contact tracing that could facilitate rapid dissemination of the respiratory virus. Humans tend to touch their eyes, nose and mouth up to about 200 times a day. When one touches a contaminated surface and then touches their face, infectious microbes are very likely to be into their body.

Studies have been conducted to determine the role of inanimate objects in vicinity of patients and/or in high viral load environments in order to quantify the role of surfaces in transmission of such pathogens. For instance, SARS-CoV-2 specimens were applied to plastic, stainless steel, copper, and cardboard and the viable virus was detected in stable condition after 72 hours of application onto these surfaces. Multiple swabs taken from COVID positive patients' rooms and from personal protective equipment of staff were found to be positive for COVID-19, although after routine cleaning of the spaces and the PPE, testing for COVID-19 was observed negative. These studies suggest that protective measures and regular decontamination of inanimate surfaces can reduce the risk of fomite transmission in different environmental settings. Currently, several professional and recreational sites like transportation, educational institutions, banks and offices, shopping centers and so on, have suffered because of the lack of knowledge about effective disinfection against such a rapidly spreading disease. It is imperative that more understanding of disinfection procedures and availability of correct resources is enhanced in order to be able to return to a normal course of livelihood. In an in-door surrounding, one can be in contact with different kinds of surface materials that can be categorized as porous or non-porous surfaces. Hard surfaces and vertical surfaces are particularly difficult to clean due to non-wetting and dripping of the disinfectant solution. The uncertainty about proper coverage through the surface, how much solution should be used, how long is the solution effective in inactivating the microbes, after how long the process should be repeated, and the like, are only some of the many open questions that need quantified explanation so as to improve the current disinfection standards. As much as, the type of disinfectant solution and its properties play a role in the process, understanding the type of material being decontaminated is also important. One finding categorizes different objects in a healthcare setting into “high touch”, “medium touch” and “low touch” based on how frequently these surfaces come in direct contact with healthcare workers and patients, thus helping in quantifying the frequency of disinfection required. The persistence of human coronavirus on inanimate surfaces like glass, copper, polypropylene, stainless steel, and so on has been found to remain infectious on such inanimate surfaces at room temperature for up to 9 days.

During this global emergency, an increase in the use of electrostatic spraying for disinfection has been observed. Several agencies have undertaken this method for safe and reliable disinfection. In electrostatic deposition technique, a potential difference is applied over disinfectant solution exiting a sprayer nozzle, so as to build electric charge near the surface of the nascent droplets as they exit the sprayer, and this charge compensates for the effects of surface tension. As a result, the droplet surface becomes unstable, which then spontaneously breaks up into smaller and mono-disperse charged droplets. The charged droplets do not coalesce in the air since they all have the same charge (like charges repel) and the droplets thus follow a trajectory to the nearest grounded surface, which is the target substrate. Since charged droplets are electrostatically attracted to the grounded object, this method exhibits a higher transfer efficiency compared to uncharged spraying methods. Recently, significant research has been conducted on the study and application of some specific types of nanoparticles, given that their inherent anti-microbial and, in some cases, anti-viral activity prevent the pathogens from generating resistance against the disinfectant, unlike many other antimicrobial solutions, which rely exclusively on the chemical disruption of the microorganisms. Different studies have confirmed that some metallic and metal oxides nanoparticles are of particular interest due to their inherent anti-microbial properties, which could also be enhanced by synergistic combinations of the particular compositions and structures. Such nanoproducts play a significant role in optimized cleaning and long-term persistence of active disinfection and inactivation of microbes on contaminated surfaces. The combination of electrostatic spraying and nano-disinfectant with the appropriate charge density, flow rate and working distance (distance of spray nozzle to target surface), are important system parameters that determine the spray characteristics and uniform deposition of anti-viral nanoparticles leading to effective disinfection process. However, the standard default system settings currently used and commercially available are not suitable for all the materials and environmental conditions. For example, the sprayer settings that are suitable for surfaces inside an aircraft cabin are not suitable for the classroom conditions. It is an indispensable need to establish reliable strategies of disinfection in order to fight the pandemic as well as continue safe practices post pandemic.

Hence there is an unmet need to establish an optimized system that can help in improving the process of existing electrostatic disinfection of porous and non-porous surfaces in various environmental settings involving medical facilities such as, but not limited to, airports, universities, and other public spaces. The present novel technology addresses this need.

### SUMMARY

Electrostatic spray deposition has been a well-known method of coating and spraying in many industries like agriculture industry, food industry, automobile industry, and so on. In 2019, when the COVID-19 pandemic started resulting in lock downs, this method of disinfection became popular and was recognized for its properties giving rise to uniform, regular, and thin coating with maximum coverage. However, ESD lacks efficacy when used on different types of surfaces and under varying environmental conditions in public and indoor settings. Currently, for aircraft cabin disinfection, 40 μm nozzle size and 7 kV of charge voltage is used for spraying which often results in dripping of disinfectant precursor solution and escalates the challenges of sterilization, thus hindering safe and adequate disinfection. The present novel technology makes use of a strategic disinfection methodology by leveraging the use of an electrostatic nebulizer with a nano-engineered disinfectant solution for reliable and effective disinfection. Scanning electron microscopy is used to support the methodology of using electrostatic deposition of nanoparticles for fighting pathogens as well as to observe the response of different surface materials to the novel spraying methodology. In experiments leading to this disclosure, the possibility of using different system parameters for electrostatic decontamination was explored and analyzed. Varying the system parameters such as different spraying distances, nozzle spray size and charge voltages allows to develop the novel methodology and to test the spraying on common, frequently touched surfaces that have been found to remain contaminated and sustain the contagious viruses longer. The observations from this study show that a nozzle spray setting of 80 μm, an optimum spray distance of eighteen inches from the substrate and three kV to seven kV of charge voltage gives substantially better results as compared to the current default disinfectant spray methodology used in the industry for standard electrostatic sprayers. It is safe to conclude that electrostatic deposition of a water-based nano-particle precursor suspension along with optimized system parameters characterized based on substrate type is superior to the known prior art techniques. The system described herein allows for nozzle changes with position feedback and without the need of manual intervention. This modification allows the system to be flexible and smart, making it efficient to be used as an autonomous and intelligent cleaning robot.

Based on the above description, it is an objective of the present disclosure to describe a surface disinfection apparatus including a tank containing a disinfectant solution; a battery-powered pump for pumping the disinfectant solution from the tank and into a nozzle capable of spraying the disinfecting onto a surface to be disinfected for a period of time; and a charge dispenser capable of ionizing the disinfectant solution prior to the disinfectant solution reaching the nozzle. In this apparatus, the charge dispenser voltage, size of the nozzle and distance between the surface and the nozzle are selectable based on the characteristics of the surface to be disinfected. It should be realized that parts of the apparatus described constitute a nebulizer. In the nebulizer, the solution containing a disinfectant or any like precursor solution is ionized to break into smaller droplets, a function of an electrostatic nebulizer. In some embodiments, the disinfectant solution is nanoxen.

In the surface disinfection apparatus of this disclosure, the surface to be disinfected can be made of one of the group consisting of wood, a plastic or polymer material, glass, metal, and combinations thereof. Examples of plastic materials suitable to be disinfected by utilizing the apparatus described and claimed herein include, but are not limited to, polyethylene plastic, PVC, polypropylene, PTFE, and rubber. Examples of metallic materials suitable to be disinfected by utilizing the apparatus of this disclosure include, but are not limited to copper, aluminum, alloys of copper, alloys of aluminum, cast iron, stainless steel, and combinations thereof. In some embodiments of the disinfection apparatus of this disclosure, the distance between the nozzle outlet and the surface is in the range 0.5 feet to 2.5 feet. In some embodiments of the disinfection apparatus of this disclosure, the spraying period is in the range of 1-3 seconds. In some embodiments of the disinfection apparatus of this disclosure, the ion generator voltage is in the range of 1 kV to 7 kV. In some embodiments of the disinfection apparatus of this disclosure, the nozzle size is changeable in the range of 40-110 microns automatically in response to an input signal.

It is another objective of this disclosure to describe a method of disinfecting a surface using the above described apparatus. The method includes providing an electrostatic nebulizer comprising an ion generator with a spray nozzle whose size is adjustable, wherein the electrostatic nebulizer can nebulize a disinfectant solution. A surface to be disinfected is selected. Further the size of the spray nozzle, the distance between the nozzle, and the charge dispensing voltage of the nebulizer are selected based on prior knowledge, namely the composition and physical characteristics of the surface to be sprayed. Those physical characteristics typically include material composition, porosity, wettability, electrical conductivity, surface roughness, and combinations thereof. The nebulizer is then actuated with the selected and/or predetermined charge dispensing voltage, nozzle size, spraying time, and/or distance between the surface and the nozzle, to yield a homogeneous and uniform spraying of the surface for the predetermined period of time leading to the disinfection of the surface. In the context of the method of this disclosure, prior knowledge includes previously obtained correlation of quality of surface coverage, droplet size, uniformity, and/or charge on surface as indicated by charge to mass ratio of the disinfectant solution on the surface as measured by a non-contact voltmeter to the type of surface to be infected. In some embodiments of the method discussed herein, the disinfectant is nanoxen. In some embodiments of the method discussed herein, the surface to be disinfected is made of one of wood, plastic/polymer, metal, and/or combinations thereof. Examples of plastic materials suitable to be disinfected by utilizing the apparatus of this disclosure include, but are not limited to, polyethylene plastic, PVC, polypropylene, PTFE, and. rubber. Examples of metallic materials suitable to be disinfected by utilizing the apparatus of this disclosure include, but are not limited to copper, aluminum, alloys of copper, alloys of aluminum, cast iron, stainless steel, and combinations thereof. In some embodiments of the method the distance between the nozzle outlet and the surface is in the range 0.5 feet to 2.5 feet. In some embodiments of the method, the spraying duration is in the range of 1-3 seconds. In some embodiments of the method, the ion generator voltage in the nebulizer is in the range of 1 kV to 7 kV. In some embodiments of the method, the nozzle size is automatically changeable in the range of 40-110 microns by a servo operationally connected to a microprocessor in response to a remotely generated input signal.

### BRIEF DESCRIPTION OF DRAWINGS

While some of the figures shown herein may have been generated from scaled drawings or from photographs that are scalable, it is understood that such relative scaling within a figure are by way of example, and are not to be construed as limiting.

FIG. 1 shows action of nanoxen on pathogens.

FIG. 2 shows an electrostatic sprayer according to a first embodiment of the present novel technology.

FIG. 3 is a schematic representation of one setup of electrostatic spray deposition (ESD) for varying system parameters.

FIG. 4 shows a camera image of a Polytetrafluoroethylene (PTFE) surface for non-ESD (left) and ESD (right).

FIG. 5 is an SEM image of a stainless-steel surface for non-ESD (left) and ESD (right).

FIG. 6 shows an SEM image of polyethylene surface for non-ESD (left) and ESD (right).

FIG. 7 shows an SEM image of a PTFE surface for non-ESD (left) and ESD (right).

FIG. 8 shows SEM image of a PVC surface for non-ESD (left) and ESD (right).

FIG. 9 shows an SEM image of polypropylene surface for non-ESD (left) and ESD (right).

FIG. 10 shows an SEM image of wood surface for non-ESD (left) and ESD (right).

FIG. 11 graphically illustrates the differential electric field as a function of nozzle distance; working distance as a function of nozzle diameter; and voltage as a function of working distance for wooden surfaces.

FIG. 12 graphically illustrates the differential electric field as a function of nozzle distance; working distance as a function of nozzle diameter; and voltage as a function of working distance for polyethylene surfaces.

FIG. 13 graphically illustrates the differential electric field as a function of nozzle distance; working distance as a function of nozzle diameter; and voltage as a function of working distance for aluminum surfaces.

FIG. 14 graphically illustrates the differential electric field as a function of nozzle distance; working distance as a function of nozzle diameter; and voltage as a function of working distance for PVC surfaces.

FIG. 15 graphically illustrates the differential electric field as a function of nozzle distance; working distance as a function of nozzle diameter; and voltage as a function of working distance for cast iron surfaces.

FIG. 16 graphically illustrates the differential electric field as a function of nozzle distance; working distance as a function of nozzle diameter; and voltage as a function of working distance for stainless steel surfaces.

FIG. 17 graphically illustrates the differential electric field as a function of nozzle distance; working distance as a function of nozzle diameter; and voltage as a function of working distance for rubber surfaces.

FIG. 18 graphically illustrates the differential electric field as a function of nozzle distance; working distance as a function of nozzle diameter; and voltage as a function of working distance for copper surfaces.

FIG. 19 graphically illustrates the differential electric field as a function of nozzle distance; working distance as a function of nozzle diameter; and voltage as a function of working distance for polypropylene surfaces.

FIG. 20 graphically illustrates the differential electric field as a function of nozzle distance; working distance as a function of nozzle diameter; and voltage as a function of working distance for PTFE surfaces.

FIG. 21 graphically illustrates the differential electric field as a function of nozzle distance; working distance as a function of nozzle diameter; and voltage as a function of working distance for wooden surfaces.

FIG. 22 graphically illustrates the differential electric field as a function of nozzle distance; working distance as a function of nozzle diameter; and voltage as a function of working distance for first aircraft surfaces.

FIG. 23 graphically illustrates the differential electric field as a function of nozzle distance; working distance as a function of nozzle diameter; and voltage as a function of working distance for second aircraft surfaces.

### DETAILED DESCRIPTION

For the purposes of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated device, and such further applications of the principles of the disclosure as illustrated therein being contemplated as would normally occur to one skilled in the art to which the disclosure relates.

The wide spread of COVID-19 pandemic has made it imperative that strategic disinfection of surfaces is practiced to fight the coronavirus and its variants. Herein, commonly contaminated surfaces such as plastics and polymers, metals, and wood are entertained for observation as to how such surfaces respond to electrostatic spraying of nano-particle based disinfectant solution. Surface based characterization is important because when different surface materials are sprayed with nano-solution and observed under a scanning electron microscope, it is seen that the nanoparticle distribution varies under same spray conditions. This means that based on the type of surfaces, the effectiveness of spray changes. To test this, twelve different surfaces were examined, including samples from inside an aircraft, with a range of system parameters for an electrostatic spray system and it was observed that most of the sample surfaces, when sprayed from a distance of eighteen inches with an 80 μm spray nozzle and at an ion generator voltage between 3-7 kV the qualitative and quantitative results show more uniform and optimized distribution of micro-droplets of precursor solution. These results are unlike the system settings currently used by disinfection industry in different sectors.

The global infection control community states that contaminated environmental surfaces play an important contributing factor to transmission of several pathogens. Different respiratory viruses including human coronavirus can remain active and infectious on inanimate surface at room temperature for several days. Some studies show quantitative observations on how frequently some objects are touched when in contact with patients and medical providers in healthcare settings—such frequent contact yields highly contaminated surfaces, while some studies have highlighted the persistence of human coronavirus on different metals, polymers and wood. These materials are present around us, in common public places like classrooms, offices, airports, hospitals, restaurants, gym and in almost any indoor setting. Ten surfaces were examined that compose different levels of porosity, and are a potential route of virus transmission, either by touch or if someone coughs or sneezes around them (see Table 1). Referring to Table 1, the Aircraft 1 sample is an aircraft-sourced decorative laminate on a phenolic composite stackup and Aircraft 2 sample is an aircraft-sourced polyurethane topcoat. Aircraft 1 and 2 are materials used in and harvested from an airplane interior cabin.

TABLE 1
Selected samples based on persistence of virus on the surface material

| Surface Samples | Category |
|---|---|
| Copper | Metal |
| Aluminum | Metal |
| Cast Iron | Metal |
| Stainless Steel | Metal |
| Polyethylene Plastic | Polymer |
| PVC | Polymer |
| Polypropylene | Polymer |
| Teflon | Polymer |
| Rubber | Polymer |
| Aircraft 1 | Polymer |
| Aircraft 2 | Polymer |
| Wood | Wood |

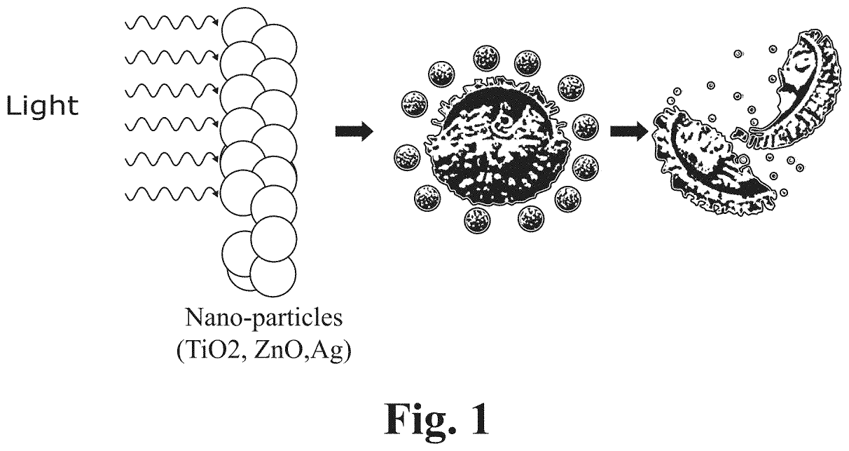

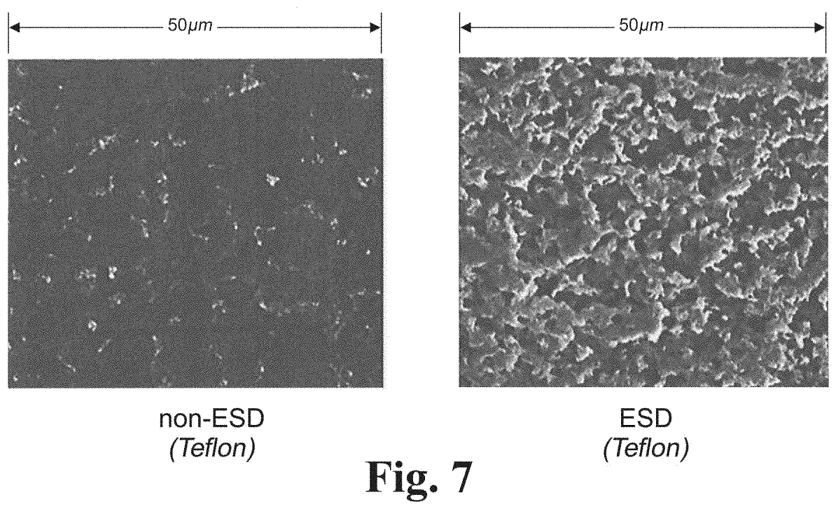

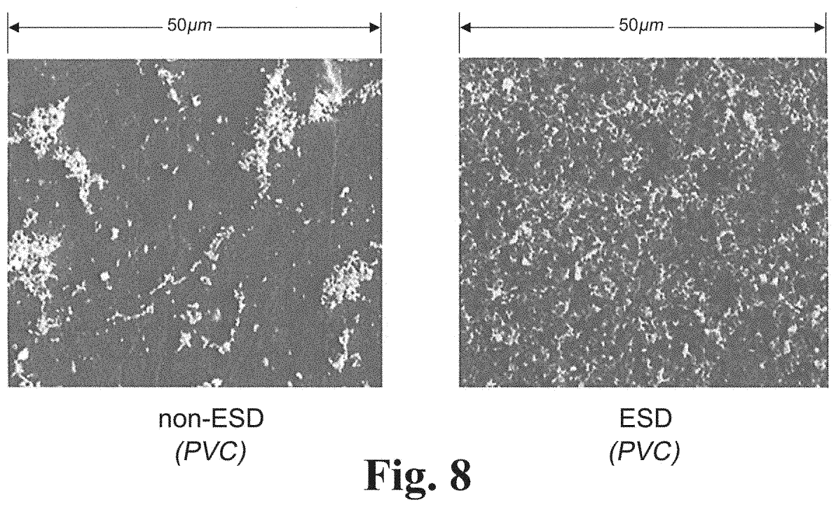

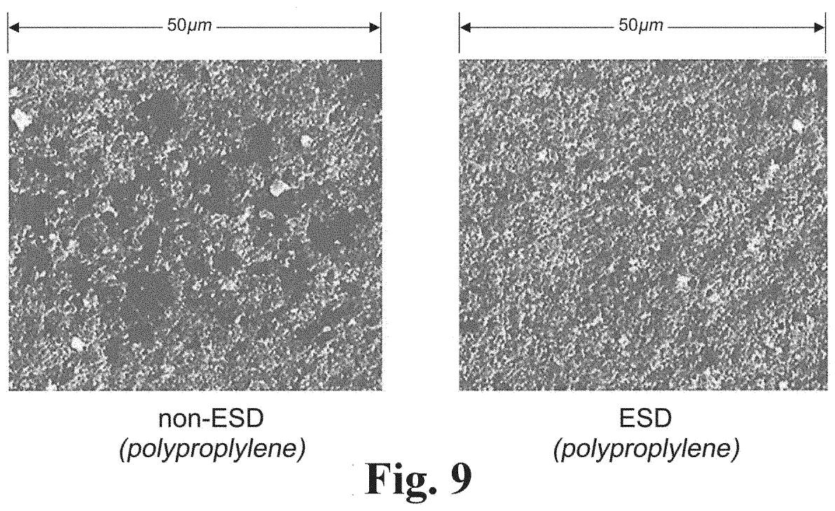

FIGS. 1-22 illustrate one embodiment of the present novel technology, an electrostatic sprayer assembly with charge-variable ion generator for providing variable voltage to a liquid disinfectant and a means, such as a gear key system, for automatically changing the spray opening size of a nozzle operationally connected to the liquid disinfectant reservoir, typically through remote control. The assembly was used to spray a nanodisinfectant on ten common surfaces that were identified as frequently touched. The surface samples were observed under a scanning electron microscope to compare between charged and uncharged spraying of the nanoparticle based disinfectant liquid. The surface samples were tested for different voltage (charge) discharge, flow rates, and working distance from the substrate and observations were noted.

Nanoparticle Based Disinfectant: Hereinbelow, nanoxen is used as the disinfectant liquid. Nanoxen is a recently developed nanotechnology-derived water-based suspension, claiming disinfection with both microbicidal and microbiostatic properties, by using mono and multi-component nanostructures selected from one or more suitable inorganic nanoparticles, one or more ceramic nanoparticles, and one or more carbonaceous nanoparticles suspended in a glycol-based vehicle, along with a rheology modifier and a surfactant. Such a nanoproduct offers great value during these current times of a global pandemic. The inorganics may include Ag, Au, Co, Cr, Cu, Fe, Ni, Mn, Zn, and combinations thereof. The ceramics may include zinc oxide, tin oxide, titania, silica, alumina, and combinations thereof. The carbonaceous nanoparticles may include fullerenes, diamond, carbon nanotubes (single and/or double walled), graphene nanoplates, graphene oxide, reduced graphene, and combinations thereof. Generally, the composition of nanoxen includes nanoparticles with a photocatalytic behavior. The disinfection properties of photocatalysis at the nanoscale is attributed to the generation of reactive oxygen species on the surface of the nanoparticles. Furthermore, the disinfection capacity and overall performance of photocatalysts may be significantly improved through surface, shape, and size modifications of the photocatalytic nanomaterial. Also, the interaction of light of the entire UV through IR electromagnetic spectrum with the nanostructures results in the formation of free metal ions. In general, the bactericidal and anti-viral activity of nanoparticles are known to depend on size, stability, and concentration in the growth medium, since while growing in a medium modified or added with nanoparticles, the microorganisms population growth can be inhibited by some specific nanoparticle interactions. For typical bacteria and virus, the cell size lies in the micrometer range, whereas their outer cellular membranes (or coatings) have pores in the nanometer range. This makes nanoparticles suitable for crossing the cell membrane/viral coating so as to penetrate the same and thus produce physiocatalytic disruption of the pathogen structure. This is in contrast with the generation of a purely chemical reaction, as it is the case of standard disinfectants, making compositions like nanoxen very efficient for disinfection. Additionally, a much lower concentrations of nanoxen is required to effectively annihilate a population of microorganisms, thus decreasing toxicity effects. Photocatalytic nanostructures have been broadly used for killing different families of microorganisms including bacteria, fungi, lichens and viruses, because photocatalytic nanostructures present high photoreactivity, broad-spectrum antibiosis, and chemical stability while used on different surfaces. This allows for the decomposition of organic compounds by the formation and constant release of hydroxyl radicals and superoxide ions when exposed to light. These radicals and superoxides are highly efficient in inhibiting the growth of even antibiotic-resistant microorganisms (see FIG. 1). The so-treated surfaces are subject to wear over time, mostly due to direct contact or environmental factors. Understanding the substrate-particle adhesion for uniform and proper deposition of anti-microbial nanoparticles is helpful. Specifically, such an understanding helps in substantiating that the anti-viral property of the solution sprayed on the surface remains active for long periods of time and is effective. In the electrostatic spray deposition (ESD) process, electrostatic image force of attraction and Coulombic force of repulsion play predictable roles in the deposition of spray film and may control the maximum coverage over surface of different shapes and sizes. The electrostatically charged nanoparticles, after initial deposition on the grounded substrate, repel the new incoming particles that in turn tend to form a layer away from the existing charged particles, spreading evenly and homogeneously around the surface. This effect assists in avoiding agglomeration of the nanoparticles, which is common when sprayed using traditional spraying technique.

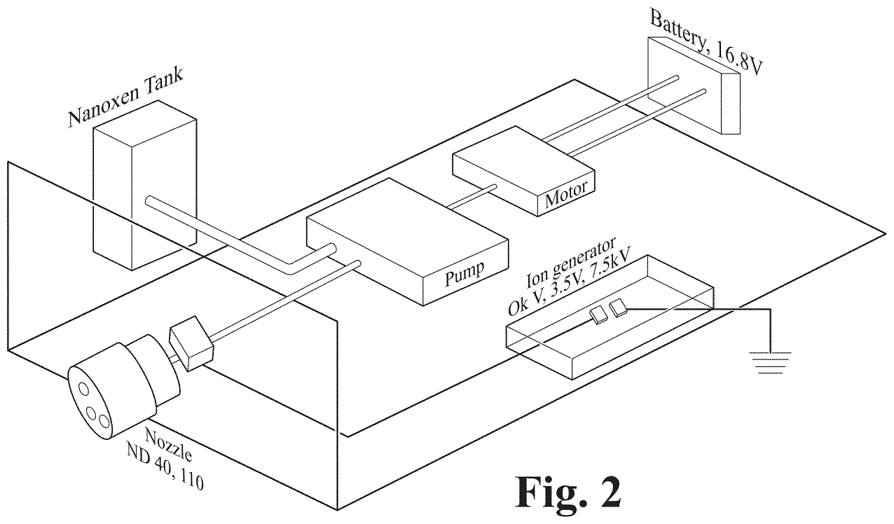

Prototype Design: As shown in FIG. 2, a prototype electrostatic jet sprayer comprising two predetermined functions was used for spray deposition of the disinfectant. The first function is to selectively vary the charge applied to the disinfectant prior to spraying. The charging module of the sprayer works in two steps. The sprayer initially charges a charging ring positioned at the tip of the sprayer and then works backwards to charge the contents of the tank. The disinfectant is then pumped through the nozzle where it is charged a second time via the charge ring before it is atomized and sprayed. The built-in charging module (also called an electric charge source), which is typically a positive ion generator, applies a potential difference of +7 kV. The casing of the sprayer is removable, allowing for changeout of the charging module. A separate positive ion generator capable of applying a potential differential of 3.5 kV was operationally connected to the sprayer. A circuit was constructed allowing switching between the 3.5 kV and 7 kV ion generators, which in turn allows variation of the charge applied to the spray. The 3-in-1 nozzle of the sprayer allows for the selection of spray particle sizes between 40, 80, and 110 microns. This process is traditionally achieved by using a key to manually turn the nozzle to the desired setting, but the instant system eliminates the manual element and introduces an automatic toggling function governed by a micro-controller operationally connected thereto in electric communication therewith. The stock key was redesigned so that its circumference serves as a spur gear. A pinion gear driven by a stepper motor was operationally connected to the nozzle and placed in constant mesh with the redesigned key. A microcontroller (also called a microprocessor and/or an electronic controller) was operationally connected to the stepper motor for sending signals to turn the stepper motor in 120 degrees increments to toggle between spray nozzle sizes.

Scanning Electron Microscopy: The ten surface samples of 1″×1″ dimensions were each sprayed using the prototype sprayer with voltage ON from a working distance of eighteen inches for ten seconds. Voltage ON indicated that the bipolar ion generator is set with default 7 kV output that charges the precursor nanosolution. Another set of ten test samples were sprayed with voltage OFF (0 kV ion generator output) under same conditions. The mass flow rate was 1.5 g/s at forty (40) microns nozzle size with a conical spray pattern. The samples were coated with a conductive material and prepared for SEM imaging under conditions listed in Table 2. The samples were incubated for twenty-four (24) hours at room temperature and surface scanning was done using field emission scanning electron microscopy at 10 kV acceleration voltage. High vacuum imaging is used for high magnification and resolution.

TABLE 2
Sample preparation for SEM imaging

| Test Samples | Copper, Aluminum, Cast Iron, Stainless | |
|---|---|---|
| | Steel, Acrylic, PVC, Polypropylene, Teflon, | |
| | Rubber, Wood | |
| Sample Size | 1″ × 1″ | |
| Nozzle Size | 40 | micrometer |
| Flow Rate | 1.5 | grams/second |
| Voltage | ON OFF | |
| Ion Generator Output | 7 | kV |
| Working Distance | 1.5 | feet |
| Time of Spray | 10 | seconds |

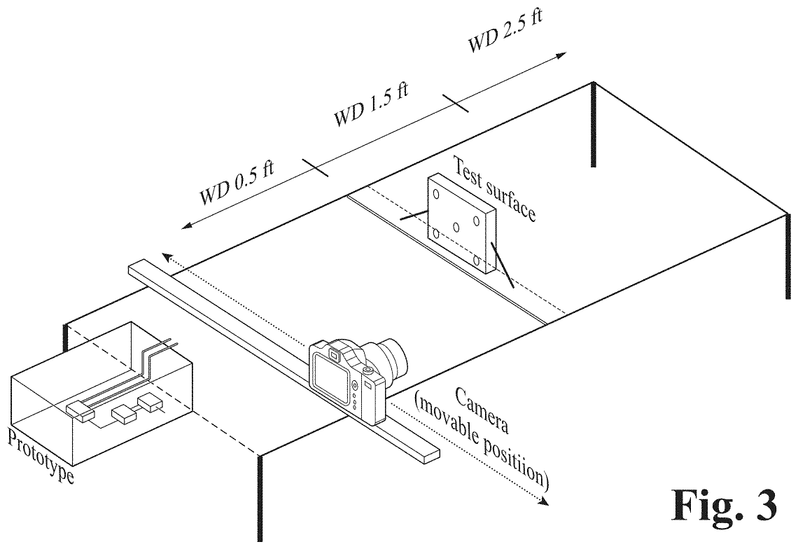

Experiments were conducted out to observe the effects of varying electrostatic spray system parameters on the different sample surfaces in a laboratory setting. The experiments included using the prototype to spray the nanodisinfectant solution on 6″×6″ sample surfaces fastened vertically to the test rig for collecting qualitative and quantitative data. The nozzle was mounted at a fixed position on one end of the test rig (see FIG. 3). The test sample is attached to a movable beam allowing us to change the distance between the nozzle and surface. The materials studied are wood, polyethylene, aluminum, PVC, cast iron, stainless steel, rubber, copper, polypropylene, PTFE, Aircraft 1, and Aircraft 2. The independent variables are: i) working distance (WD): distance between the sprayer nozzle and the plate (0.5 ft, 1.5 ft, 2.5 ft), ii) charging voltage (0 kV, 3.5 kV, 7 kV), and iii) spray particle size (40 micron, 80 micron, 110 micron). For each test, the plate was first positioned upright at the same horizontal level as the nozzle at respective WD. The electric potential is measured at five (5) points using a non-contact surface voltmeter. Next, the prototype was used to spray the disinfectant for three (3) seconds. The electric potential on the substrate is measured again immediately after spraying, at five locations. Differential electric field (DEF) is calculated as the difference in the average voltmeter reading before and after spray, for the five (5) points on the sample surface. An image of the droplet pattern is obtained using a mounted camera. The test surface was then removed, wiped, and allowed to discharge over a period of time. This was repeated for every combination of the independent variables for each of the twelve test materials. The quantitative measurement of the electric field potential was done at a fixed distance, at close proximity, about two (2) mm to the surface in normal direction. Charge to mass ratio is a crucial parameter that is used to determine the effectiveness of electrostatic spray deposition. This can be represented as a ratio of charge (Coulomb) to flow rate (kg/s). The electric potential at a point is directly proportional to charge and inversely proportional to the square of distance between the charge and origin. Since the non-contact voltmeter has negligible error in measurement as the normal distance between the surface and voltmeter probe is increased, the distance can be treated as constant, making the electric field proportional to the charge. The observation made using the non-contact voltmeter can be used to measure the charge to mass ratio. Herein, results are presented in the form of differential electric field (DEF) compared for different cases as shown in Table 3:

TABLE 3
Test matrix with varying voltage (V), nozzle
spray size (ND) and working distance (WD)

| Independent | | | |
|---|---|---|---|
| Variables | Case Matrix | | |
| Spray | 40 μm, | 80 μm, | 110 μm, |
| Particle Size | cone spray | cone spray | fan |
| Working | 0.5 feet | 1.5 feet | 2.5 feet |
| Distance | | | |
| Charging | OkV | 3 ± 0.5 | 7 ± 0.5 |
| Voltage | | | |

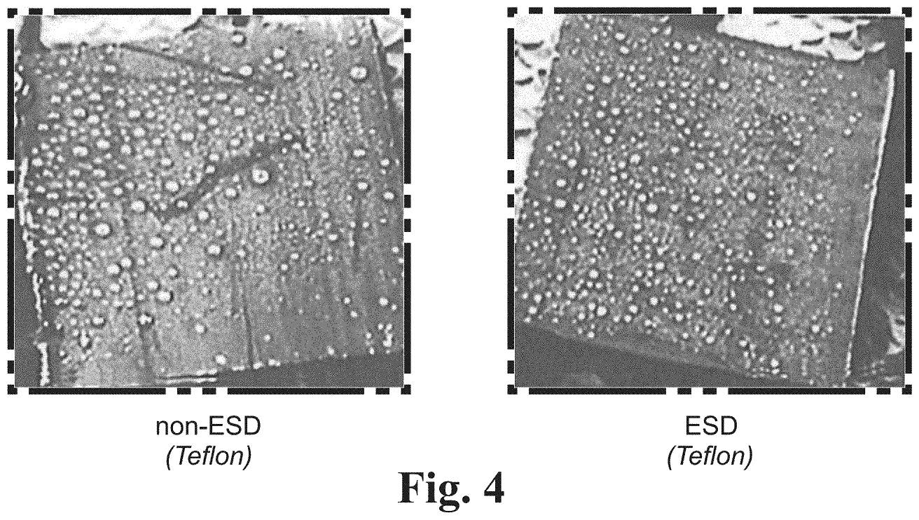

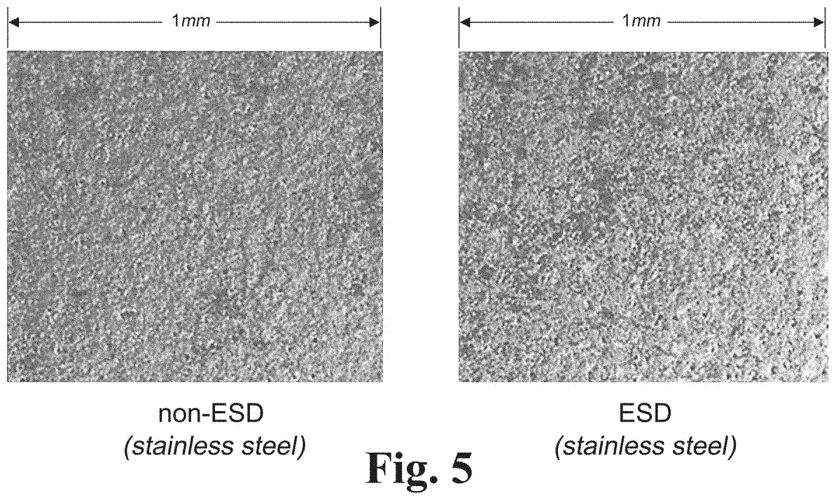

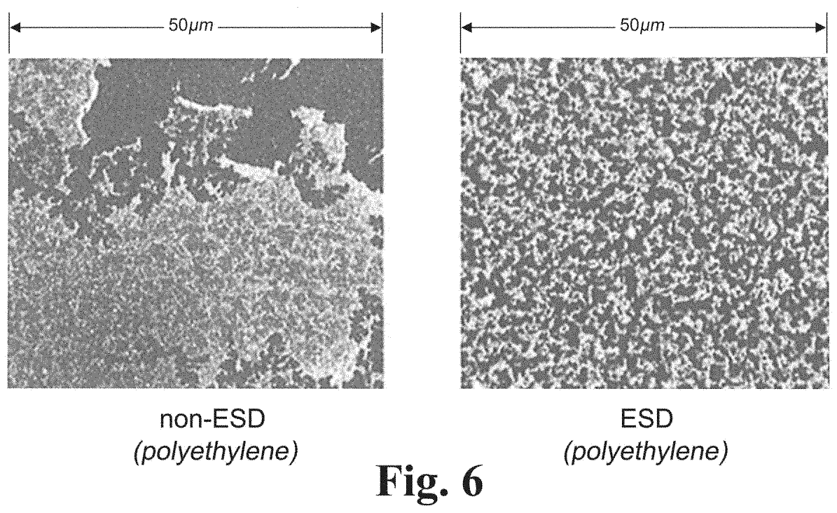

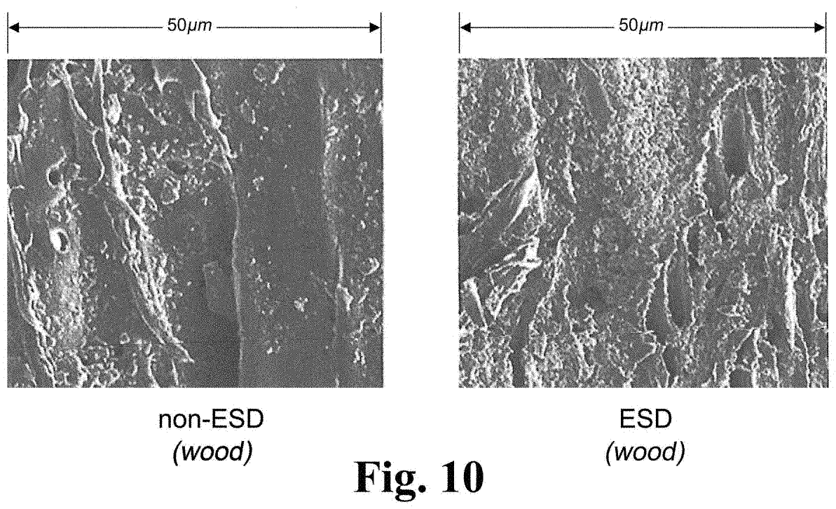

SEM images of electrostatic vs traditional spray deposition: FIG. 4 illustrates camera images of a PTFE sample coated using electrostatic spray deposition (ESD) and non-electrostatic spray. The macroscopic droplets are more uniform in size and evenly spread with the ESD as compared to the non-ESD spray. As shown in FIG. 5, back scattered SEM images of stainless steel give rise to the observation that the surface becomes substantially completely covered with the nanoparticle solution in ten (10) seconds in both the ESD and non-ESD cases, as a thick film of nanoparticles distributed throughout the metal surface is observed. Similar nature is observed for aluminum, copper and cast iron surfaces. There is dripping and over-layering of nano-solution observed on the metal surfaces. Plastics and polymers like acrylic, PTFE, PVC, and polypropylene show a distinct difference between the ESD and non-ESD coating processes, as nanoparticles form a uniform layer with larger coverage in case of electrostatic deposition (see FIGS. 6, 7, 8, and 9, respectively). On wood, due to its highly porous surface, the nano-particles get deposited in a non-uniform fashion whether deposited by ESD or non-ESD techniques; however, more clustering and agglomeration is observed in non electrostatic deposition (see FIG. 10). Twenty-four (24) hours after the application of nanoxen, nanoparticles are observed on the surfaces in the back scattered images, and effective deposition is observed in case of the electrostatic spraying technique. These qualitative observations underscore the need to vary system parameters when spraying different types of surface materials, as the default setting of voltage, flow rate and nozzle size as widely used in the known disinfection applications are not optimized parameters for safe and effective spraying and may be contributing factors to the spread of the pathogens.

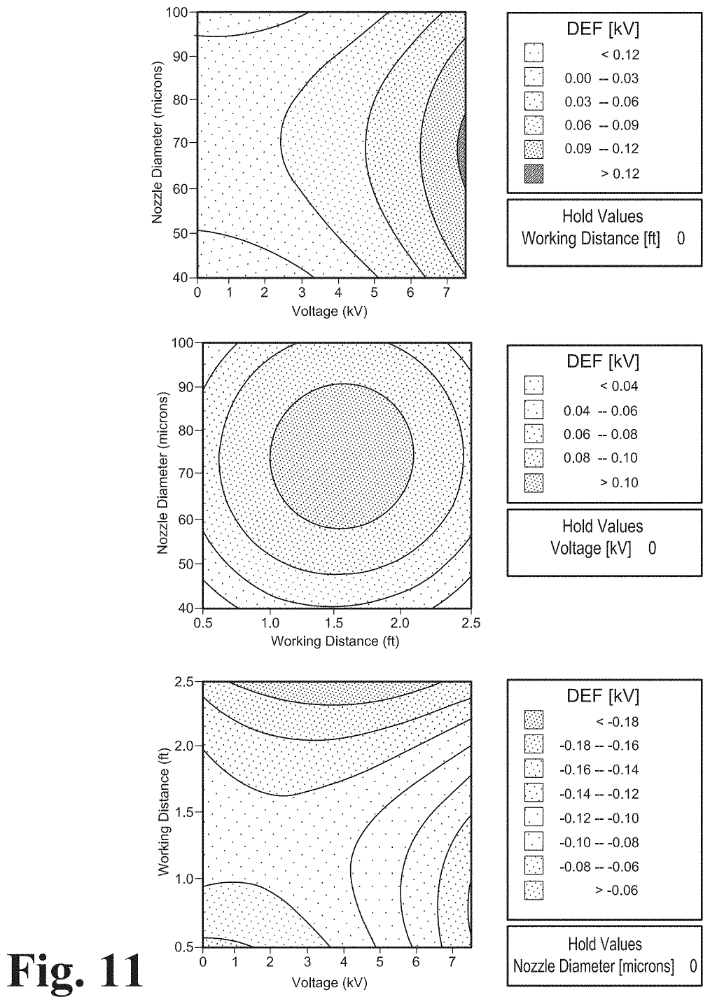

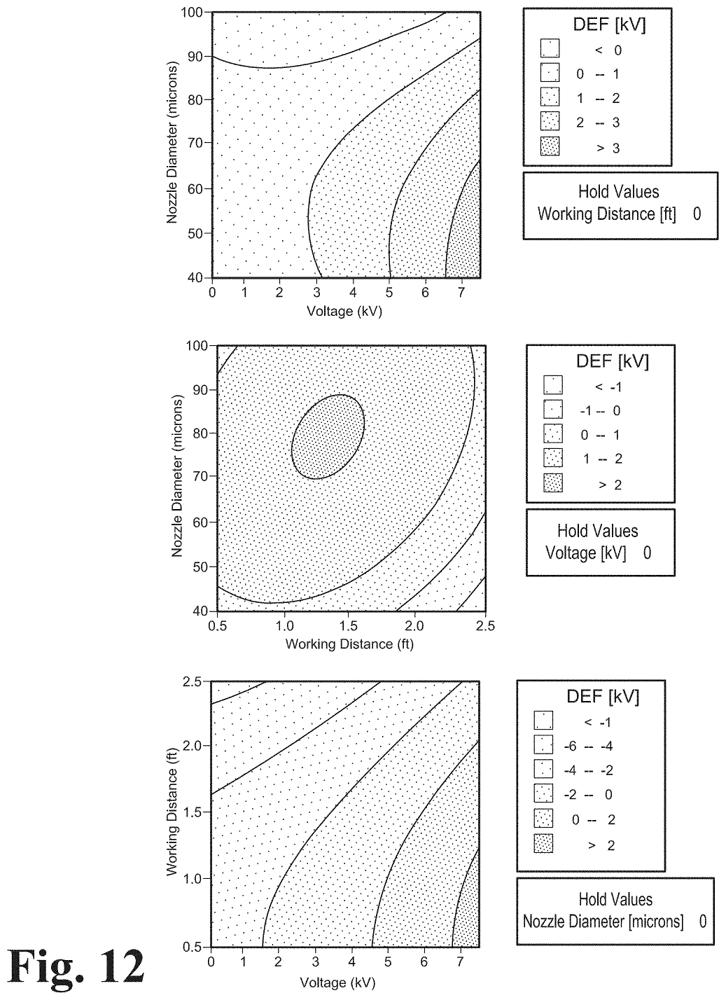

Effect of varying voltage, nozzle size and working distance: The images observed under scanning electron microscopy show the need to create a more optimized system for strategic disinfection. On varying the system parameters such as the ion generator voltage, spray nozzle diameter and the working distance of spray, for different cases we are able to see how different surfaces respond to electrostatic deposition, both qualitatively and quantitatively. Observations for each surface may be presented in the form of images and surface plots. For example, for wood, as shown in FIG. 11, qualitative images show that the surface is completely wet in 3 seconds of spraying and does not have any distinct droplets visible due to its porous nature. Dripping is observed and all the cases look qualitatively similar. From the plots in FIG. 12, it is observed that around eighteen (18) inches of WD, 80 microns of spray nozzle, and an applied charge above 6 kV gives a high DEF, which corresponds to better charge to mass ratio as compared to other settings.

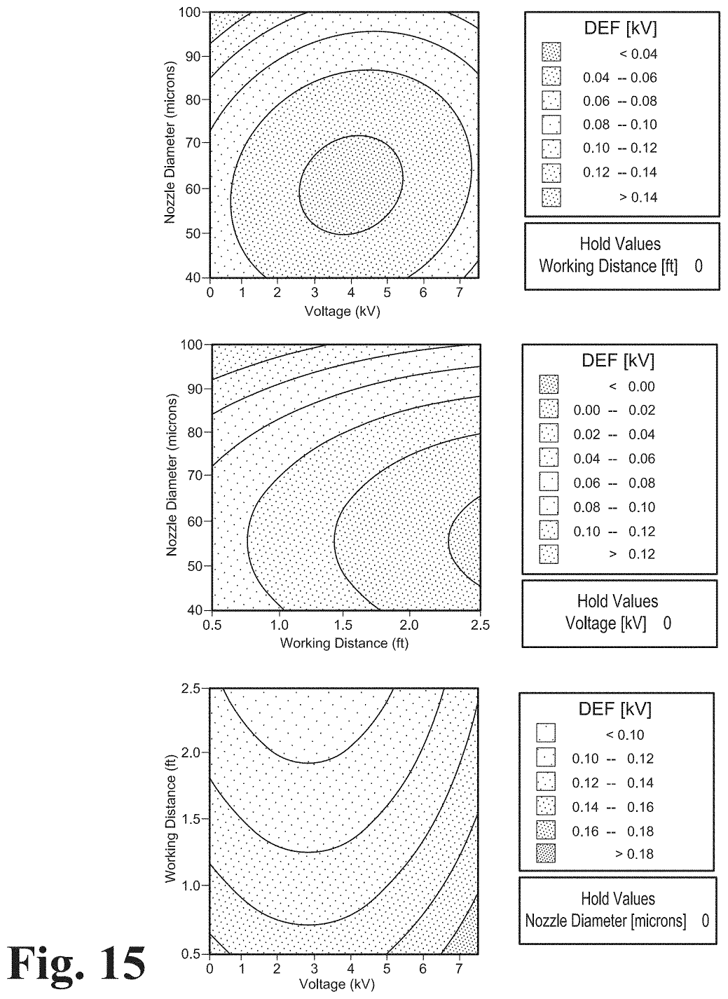

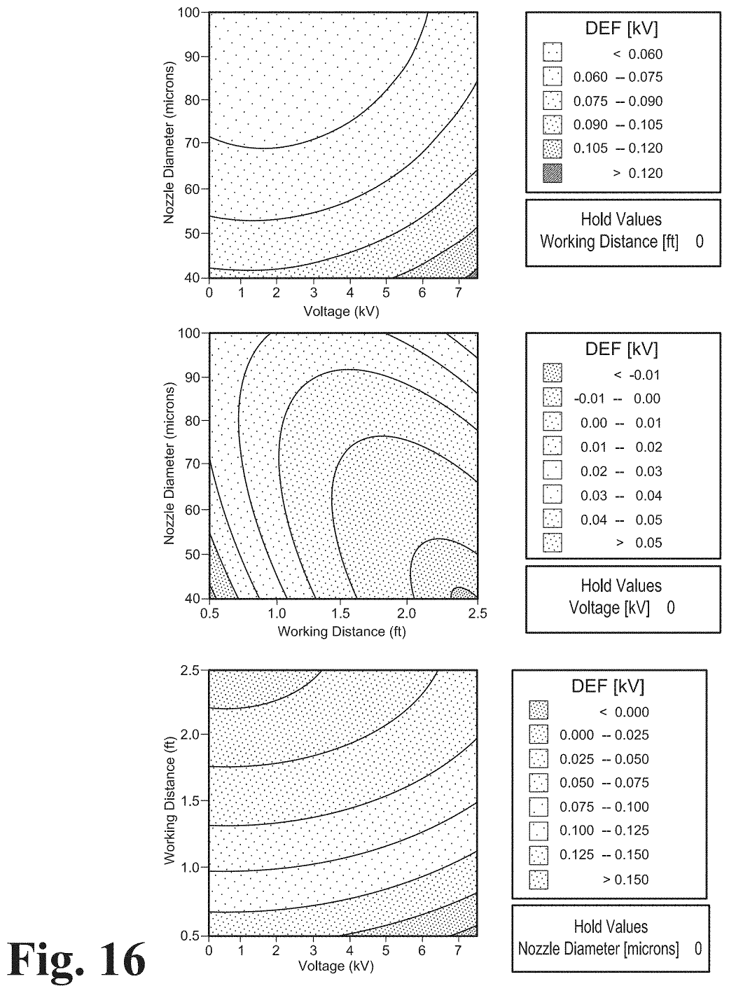

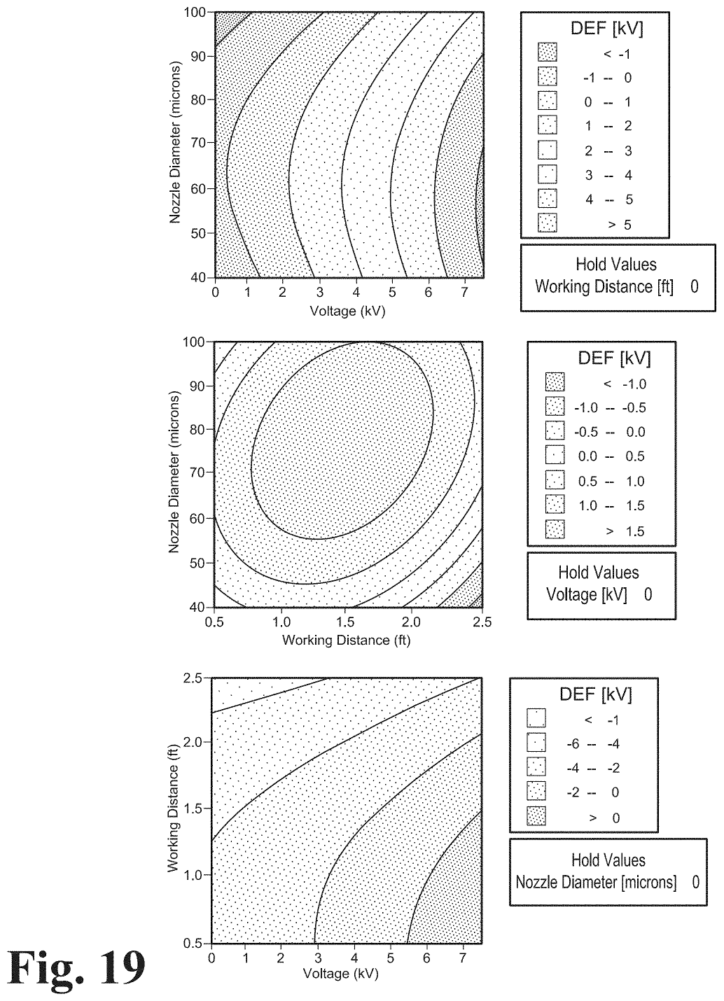

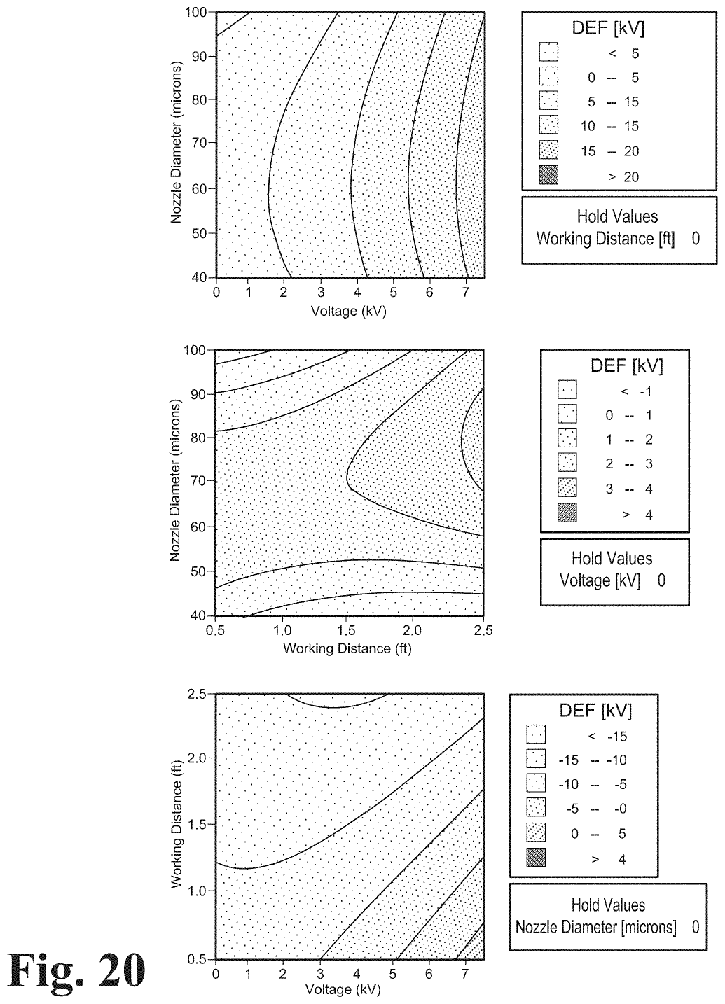

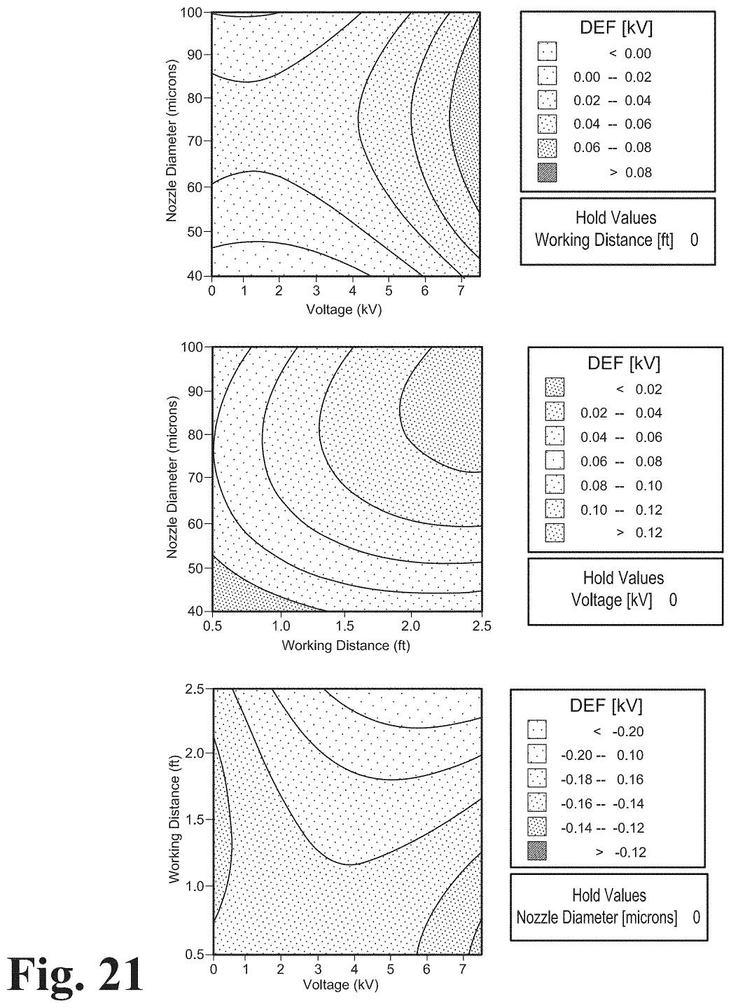

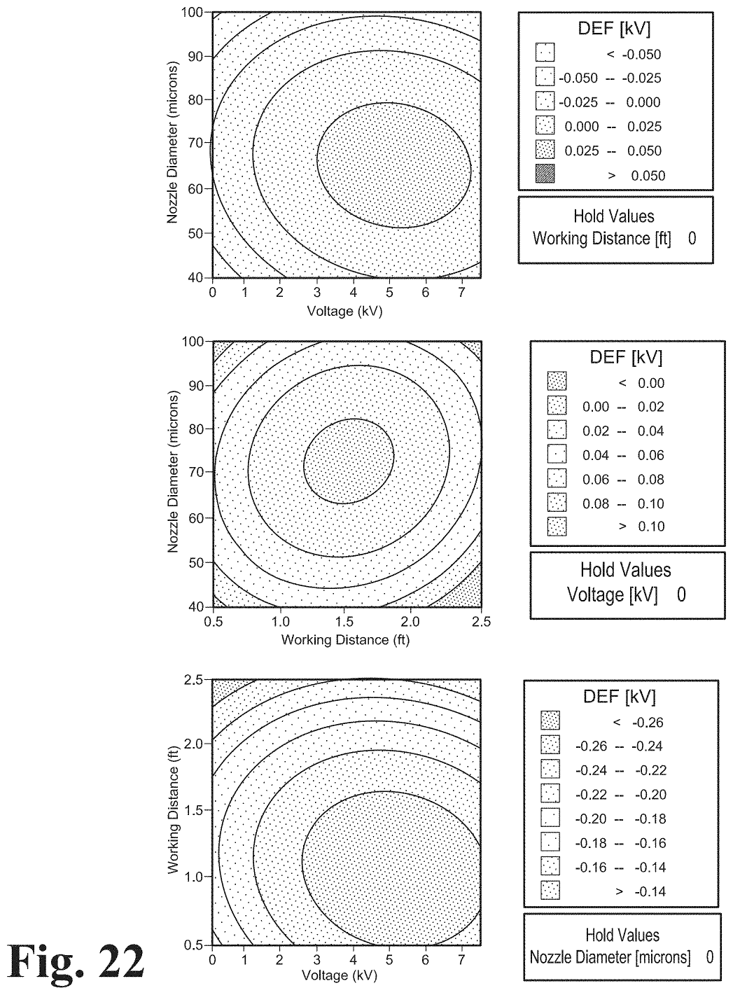

For aluminum, as shown in FIG. 15, it is observed that spraying at a distance between eighteen (18) and thirty (30) inches, at 80 microns spray size yields good coverage as well as uniform droplet size and distribution on the surface. Quantitatively, as shown in FIG. 16, it is observed from the plots that the effect of voltage is negligible, since the metal surface does not retain much charge on its own. Here we observe a contrast as compared to the qualitative results and see that the best region of effective spray lies for 2-7 kV, 40-60 microns nozzle dimension, and between six (6) inches and eighteen (18) inches of working distance. For copper, as shown in FIG. 25, similar to the other metals it is observed that the voltage change does not make much difference; however changing the working distance and nozzle diameter shows a more substantial difference. Uniform spread is observed at 80 microns nozzle dimension and eighteen (18) inches working distance. Quantitative results given in FIG. 26 show that at 80 microns setting, a working distance of thirty (30) inches results in higher DEF, independent of the voltage setting. For cast iron, shown in FIG. 19, no distinct droplets are observed while most of the disinfectant drips off the surface. There are large droplets mostly accumulated on the lower end of the vertical surface. As shown quantitatively in FIG. 20, the value range of DEF is almost negligible. High nozzle spray diameter is observed to be resulting in higher DEF as compared to other conditions. For stainless steel (see FIG. 21) non-uniform droplet distribution and accumulation at the lower half of the surface is observed, caused due to dripping of the nano-solution; this is mirrored in all metallic substrate cases. From plots as shown in FIG. 22, it is seen that the value range of DEF is almost negligible, and a small working distance and nozzle spray size gives higher DEF.

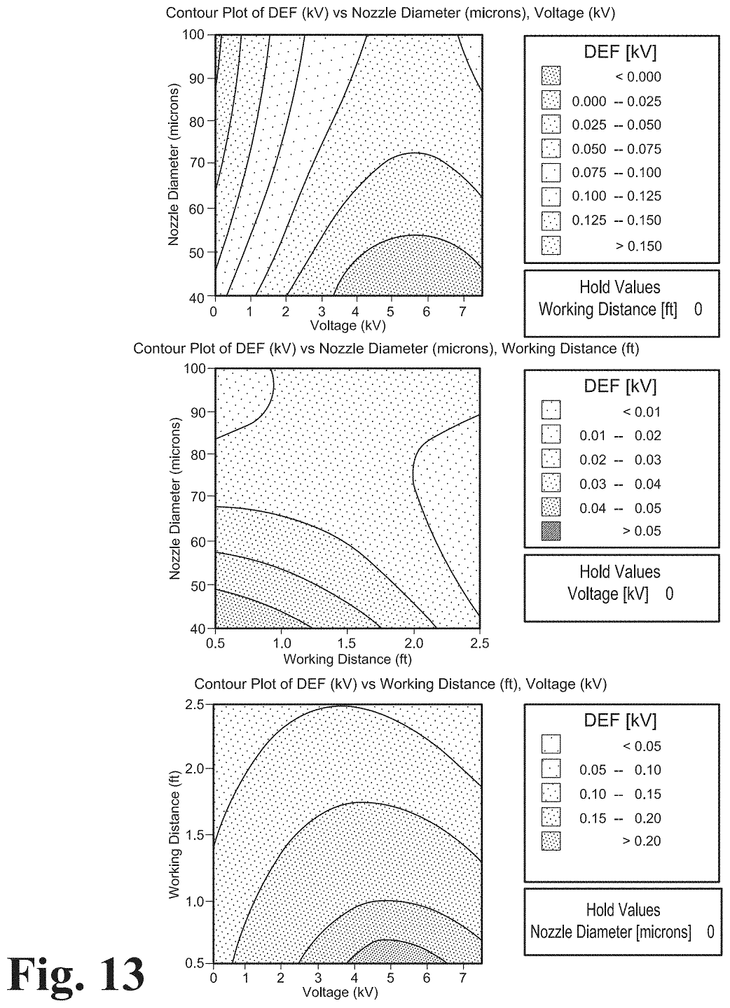

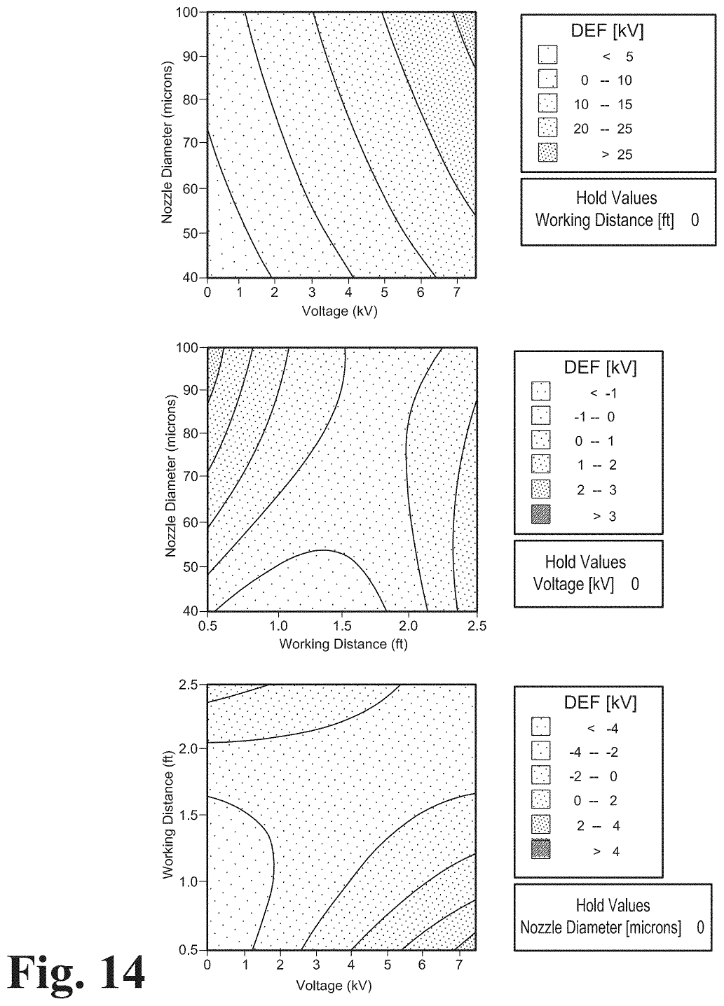

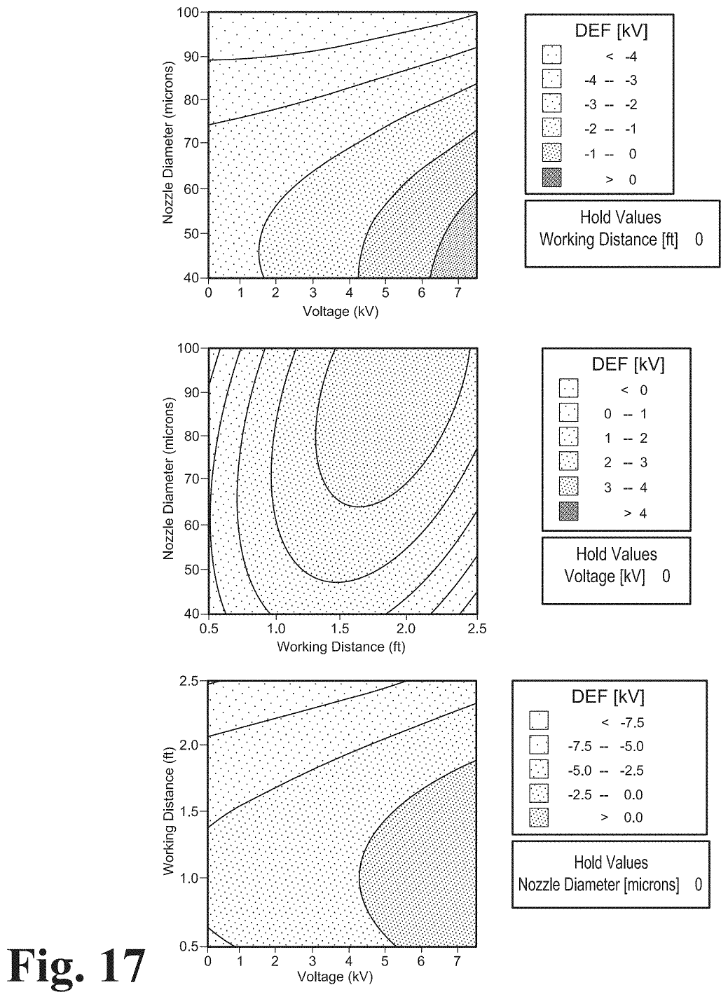

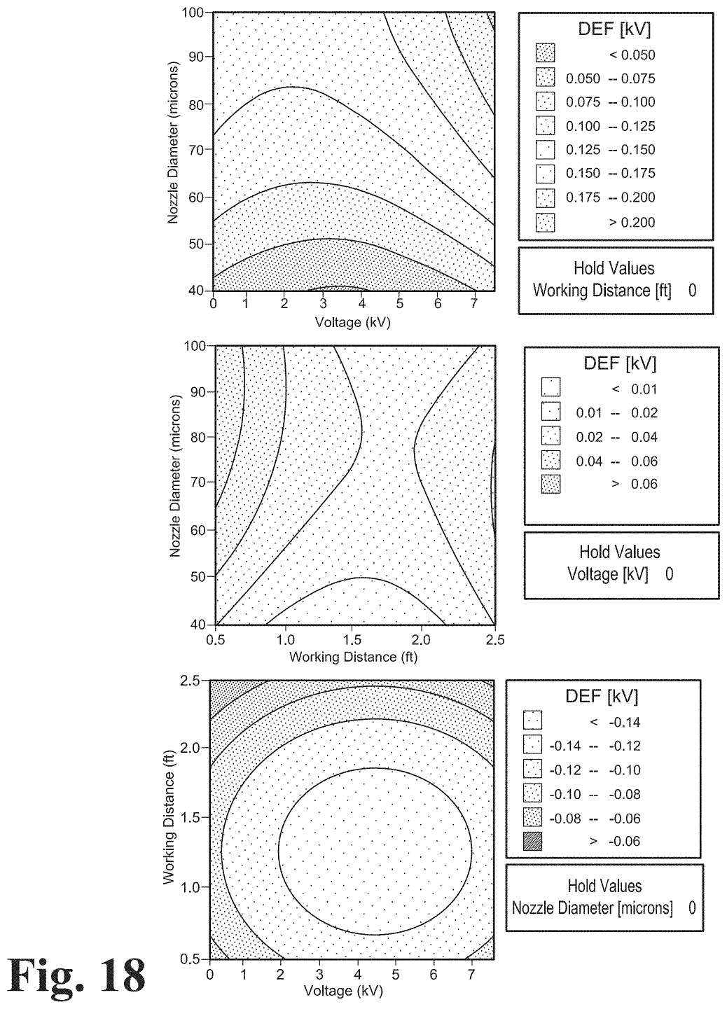

For a polypropylene substrate (see FIG. 27), it is observed that qualitatively at WD of eighteen (18) inches and 80 microns spray setting, proper droplet distribution is observed, independent of the applied voltage. The plots (see FIG. 28) show agreement with the qualitative results at high voltage setting. For the polyethylene plastic substrate (see FIG. 13), it was observed that at WD eighteen (18) inches and 80 microns system setting, uniform droplet distribution is observed. This is in agreement with the DEF surface plots (see FIG. 14) at high charge voltage. For PTFE substrate (see FIG. 29), qualitative results show that eighteen (18) inches working distance and 80 microns nozzle spray size, as well as thirty (30) inches working distance at 110 microns setting, show uniform spread. From surface plots (see FIG. 30), a range of nozzle setting between 50-80 microns at high working distance while higher voltage setting is shown to be more effective. For the PVC substrate shown in FIG. 17, similar qualitative nature as for the PTFE substrate was observed. Quantitatively (see FIG. 18) either a high voltage with low working distance or low voltage and high working distance, with all nozzle sizes working well at higher voltages, is observed. On a rubber surface (shown in FIG. 23) wetting and dripping was observed with no distinct droplets except at highest nozzle size and working distance. Quantitatively (see FIG. 24) all the three parameters at the higher end give maximum DEF. For the samples from aircraft cabin, Sample 1 (see FIG. 31) shows large droplets and dripping in most cases. From the quantitative results (see FIG. 32) it is seen that between eighteen (18) and thirty (30) inches working distance and 60-100 microns spray size as well as higher end of voltage setting results in the maximum DEF. For Sample 2 (see FIG. 33) better qualitative results are obtained at 80 microns setting and eighteen (18) inches working distance. This is in agreement with the quantitative results (see FIG. 34) at higher voltage range between 3-7 kV.

While the present disclosure has been described with reference to certain embodiments, it will be apparent to those of ordinary skill in the art that nigh-infinite other embodiments and implementations are possible that are within the scope of the present disclosure without departing from the spirit and scope of the present disclosure. Thus, the implementations should not be limited to the particular limitations described. Other implementations may be possible. It is therefore intended that the foregoing detailed description be regarded as illustrative rather than limiting.

1. A surface disinfection apparatus comprising:
a tank containing a disinfectant liquid;
a nozzle having a size variable spray opening;
a pump operationally connected to the tank and to the nozzle for pumping disinfectant from the tank into and through the nozzle to spray disinfecting onto a surface for a period of time;
an electric charge source operationally connected to the tank for ionizing disinfectant liquid;
wherein charge dispenser voltage, spray opening size, and distance between the surface and the nozzle are predetermined based on the characteristics of the surface.

2. The surface disinfection apparatus of claim 1, wherein the disinfectant is nanoxen.

3. The surface disinfection apparatus of claim 1, wherein the surface is selected from the group consisting of wood, plastic, metal, glass, and combinations thereof.

4. The surface disinfection apparatus of claim 1, wherein the distance between the spray opening and the surface is between eighteen inches and thirty inches.

5. The surface disinfection apparatus of claim 1, wherein the period of time is between one second and three seconds.

6. The surface disinfection apparatus of claim 1, wherein the electric charge source imparts a voltage between one kV and seven kV.

7. The surface disinfection apparatus of claim 1, where in the spray opening size is automatically variable between forty microns and one-hundred and ten microns in response to a remotely generated input signal.

8. A method of disinfecting a surface, comprising:
providing an electrostatic nebulizer for nebulizing a disinfectant suspension and comprising a tanks of nanoparticulate disinfectant suspension, an ion generator operationally connected to the tank for charging the nanoparticulate disinfectant suspension with a predetermined voltage, and a variable spray opening size nozzle;
selecting a surface to be disinfected;
categorizing the surface by surface type;
selecting a predetermined opening size of the variable spray opening size nozzle based on surface type;
selecting the distance between the variable spray opening size nozzle and the surface based on surface type;
selecting the predetermined voltage based on surface type; and
actuating the nebulizer with the predetermined voltage, the spray opening size, and the distance between the surface and the predetermined spray opening size; and
spraying the surface for a predetermined period of time to yield a homogeneously sprayed surface.

9. The method of claim 8 wherein the disinfectant is nanoxen.

10. The method claim 8 wherein the surface is selected from the group consisting of wood, polymer, metal, and combinations thereof.

11. The method of claim 8 wherein the distance between the variable spray opening size nozzle and the surface is between eighteen inches and thirty inches.

12. The method of claim 8 wherein the predetermined period of time is between one second and three seconds.

13. The method of claim 8 wherein the ion generator produces a positive voltage between one kV and seven kV.

14. The method of claim 8 and further comprising a microcontroller operationally connected to the variable spray opening size nozzle, wherein the variable spray opening size nozzle has an opening size that is automatically changeable between 40 microns and 110 microns in response to a remotely-generated input signal from the microcontroller.

15. A disinfectant sprayer assembly, comprising:
a tank defining a volume;
a disinfectant suspension contained within the volume;
a nozzle having a size variable spray opening;
a pump operationally connected to the volume and to the nozzle for pumping disinfectant from the volume through the spray opening to apply disinfecting to a predetermine surface having a predetermined set of physical characteristics for a period of time;
an ion generator operationally connected to the volume for ionizing the disinfectant suspension; and
a microprocessor operationally connected to the size variable spray opening;
wherein ion generator voltage, spray opening size, and distance between the predetermined surface and the spray opening are predetermined based on the predetermined set of physical characteristics of the surface.

16. The disinfectant sprayer assembly of claim 15 wherein the predetermined set of physical characteristics are selected from the group consisting of material composition, porosity, wettability, electrical conductivity, surface roughness, and combinations thereof.

17. The disinfectant sprayer assembly of claim 15 wherein the disinfectant suspension further comprises a plurality of nanostructures suspended in a glycol-based vehicle, wherein the nanostructures are selected from the group comprising inorganic nanoparticles, ceramic nanoparticles, carbonaceous nanoparticles and combinations thereof.

18. The disinfectant sprayer assembly of claim 15 wherein the distance between the size variable spray opening and the predetermined surface is between eighteen inches and thirty inches, and the ion generator produces a positive voltage between one kV and seven kV.
